(19) United States
(12) Patent Application Publication
(10) Pub. No.: US 2023/0203434 A1
(43) Pub. Date: Jun. 29, 2023

(54) SCAFFOLD, METHOD FOR PRODUCING SCAFFOLD, CELL CULTURE CONSTRUCT, METHOD FOR CULTURING CELL

(71) Applicants: CELLFIBER CO., LTD., Tokyo (JP); LEAVE A NEST CO, LTD., Tokyo (JP)

(72) Inventors: Kazuhiro IKEDA, Tokyo (JP); Lucas Siqueira TRINDADE, Tokyo (JP)

(73) Assignees: CELLFIBER CO., LTD., Tokyo (JP); LEAVE A NEST CO, LTD., Tokyo (JP)

(21) Appl. No.: 18/115,728

(22) Filed: Feb. 28, 2023

Related U.S. Application Data
(63) Continuation-in-part of application No. PCT/JP2021/032011, filed on Aug. 31, 2021.

(30) Foreign Application Priority Data
Sep. 1, 2020 (JP) 2020-147162

(51) Int. Cl. C12N 5/00; C12N 5/0775

(52) U.S. Cl. CPC C12N 5/0062; C12N 5/0068; C12N 5/0662; C12N 2513/00; C12N 2523/00; C12N 2533/56

(57) ABSTRACT

A scaffold for culturing a cell comprises: a hydrogel; and a plasma-derived or platelet-derived component or a fibrin-containing material adhered to the hydrogel.

### CROSS-REFERENCE TO RELATED APPLICATIONS

The present application is a Bypass Continuation-In-Part Application of International Patent Application No. PCT/JP2021/032011 filed on Aug. 31, 2021, which claims priority to Japanese Patent Application No. 2020-147162, filed on Sep. 1, 2020. The contents of these applications are hereby incorporated by reference in their entireties.

### TECHNICAL FIELD

The present invention relates to a scaffold for culturing cells, a method for producing a scaffold, a cell culture construct comprising the scaffold, and a method for culturing cell by using the scaffold.

### BACKGROUND ART

Currently, attempts have been made to culture various cells in various fields such as developmental biology, drug discovery, and regenerative medicine. These cells are typically cultured two-dimensionally on a surface of a plastic container for tissue culture (two-dimensional culture). In recent years, a technique for culturing cells under a three-dimensional culture environment utilizing a scaffold such as a porous membrane or a hydrogel (three-dimensional culture). The following Patent Literatures 1 to 6 disclose that cells are cultured inside a hydrogel in a form of a tube.

Here, it is known that whether or not cells are efficiently cultured depends greatly on the type of cells and the culture conditions. Culture cells that proliferate while floating in a medium are called “floating cells”. On the other hand, cells that proliferate while adhering to a scaffold are called “adherent cells”.

### CITATION LIST

### Patent Literature

Patent Literature 1: WO 2011/046105
Patent Literature 2: JP 2017-99303 A
Patent Literature 3: WO 2017/091662
Patent Literature 4: WO 2018/098295
Patent Literature 5: WO 2019/178549
Patent Literature 6: WO 2020/032221

### SUMMARY

It is known that whether or not cells are efficiently cultured depends greatly on the type of cells and the culture conditions. In particular, the efficiency of the culture of adherent cells may vary greatly depending on a scaffold for the culture. The inventors of the present application have found that in a case where a hydrogel is used as the scaffold in order to culture adherent cells, it is difficult to culture the adherent cells depending on the conditions, and there is still a room for the improvement.

Accordingly, a scaffold suitable for culturing adherent cells, a cell culture containing the scaffold, and a method for culturing adherent cells using the scaffold have been desired.

A scaffold for culturing a cell according to one aspect, comprises: a hydrogel and a plasma-derived or platelet-derived component or a fibrin-containing material adhered to the hydrogel.

According to one preferred aspect, the hydrogel has a string shape, a tube shape, a sphere shape, or a spherical shell shape.

According to one preferred aspect, the plasma-derived or platelet-derived component or the fibrin-containing material is provided an inside or outside of the hydrogel.

According to one preferred aspect, the plasma-derived or platelet-derived component or the fibrin-containing material contains a human platelet lysate-derived component.

According to one preferred aspect, the hydrogel contains an alginate gel.

According to one preferred aspect, the hydrogel contains an alginate gel, and a gelatin mixed with the alginate gel.

A cell culture construct according to one aspect comprises the above scaffold and a cell adhered to the scaffold.

According to one preferred aspect, the cell is an adherent cell.

According to one preferred aspect, the adherent cell is a mesenchymal stem cell.

A method for culturing a cell according to one aspect, comprises: forming a scaffold comprises a hydrogel and a plasma-derived or platelet-derived component or a fibrin-containing material adhered to the hydrogel; culturing a cell by adhering the cell to the scaffold.

A method for producing a scaffold for culturing a cell according to one aspect, comprises contacting a suspension containing a plasma-derived or platelet-derived component, fibrinogen or fibrin, or a mixture thereof with the alginate gel.

According to one preferred aspect, the method for producing a scaffold comprises: turning a hydrogel precursor into a gel to form the alginate gel; and immersing the alginate gel in the suspension.

According to one preferred aspect, the method for producing a scaffold comprises: flowing a hydrogel precursor around a suspension while flowing the suspension; and turning the hydrogel precursor into a gel to form the hydrogel which has a tube shape covering the suspension.

According to one preferred aspect, the suspension contains a human platelet lysate.

According to one preferred aspect, the hydrogel precursor contains an alginate solution.

According to the above aspects, a scaffold suitable for culturing adherent cells, a method for producing the scaffold, a cell culture containing the scaffold, and a method for culturing adherent cells using the scaffold can be provided.

### BRIEF DESCRIPTION OF DRAWINGS

FIG. 1 is a photograph showing a structure of the scaffold for culturing cells according to the first embodiment.

FIG. 2 is a schematic diagram showing a cross section of the scaffold for culturing cells according to the first embodiment.

FIG. 3 is a photograph showing an example of a structure of a cell culture construct having the scaffold for culturing cells according to the first embodiment and cells adhered to the scaffold.

FIG. 4 is a schematic diagram showing a structure of the scaffold for culturing cells according to the second embodiment.

FIG. 5 is a schematic diagram showing a cross section of the scaffold for culturing cells according to the second embodiment.

FIG. 6 is a photograph showing an example of a structure of a cell culture construct having the scaffold for culturing cells according to the second embodiment and cells adhered to the scaffold.

FIG. 7 is a schematic diagram showing an example of an apparatus for producing the scaffold for culturing cells according to the second embodiment.

FIG. 8 is a graph showing proliferation rates of the cells cultured in Examples 13 to 16 and Reference Example 7 .

### DESCRIPTION OF EMBODIMENTS

As a result of intensive studies, the present inventors have found a scaffold suitable for culturing adherent cells, and a method for culturing cells by using the scaffold.

The scaffold for culturing cells has a hydrogel, and a component adhered to the hydrogel. The component adhered to the hydrogel is preferably a component being adhesive to cells. The component adhered to the hydrogel may be a plasma-derived or platelet-derived component, or a fibrin-containing material.

The hydrogel may have a string shape, a tube shape, a sphere shape, or a spherical shell shape. From the viewpoint of securing the surface area of a scaffold and ease of handling the scaffold, the hydrogel has more preferably string shape or a tube shape.

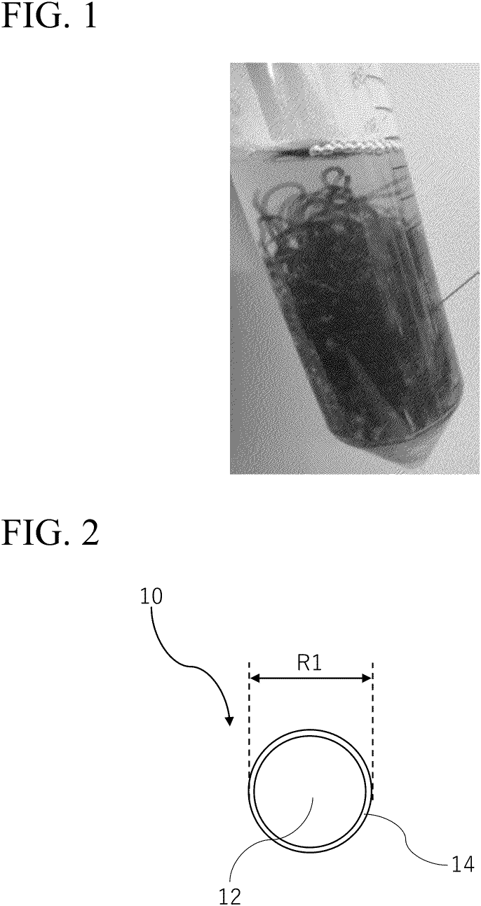

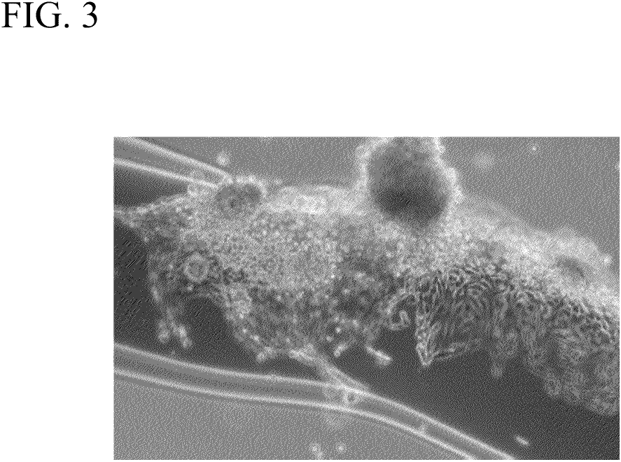

FIG. 1 is a photograph showing a structure of the scaffold for culturing cells according to the first embodiment. FIG. 2 is a schematic diagram showing a cross section of the scaffold for culturing cells according to the first embodiment. The structure with a form of a string in a container of the photograph shown in FIG. 1 is a scaffold 10. In the embodiment shown in FIGS. 1 and 2, the scaffold has a hydrogel 12 in a form of a continuously extending string. A plasma-derived or platelet-derived component, or a fibrin-containing material 14 is provided on the outer surface of the hydrogel 12 (see FIG. 2). In this case, adherent cells are cultured while adhering to a component 14 on the outer surface of the hydrogel 12 (see FIG. 3). FIG. 3 shows an enlarged portion of a cell culture construct obtained by adhering mesenchymal stem cells (MSCs) to the scaffold for culturing cells, and culturing the MSCs. Further, by continuing to culture the cells, it is possible to culture the cells from the state shown in FIG. 3 to the state of a larger number of cells.

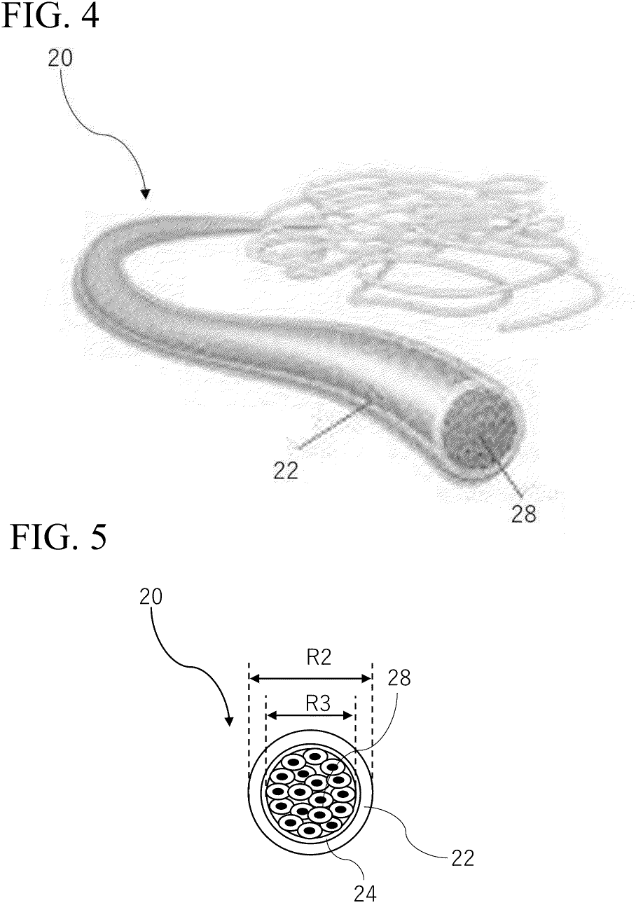

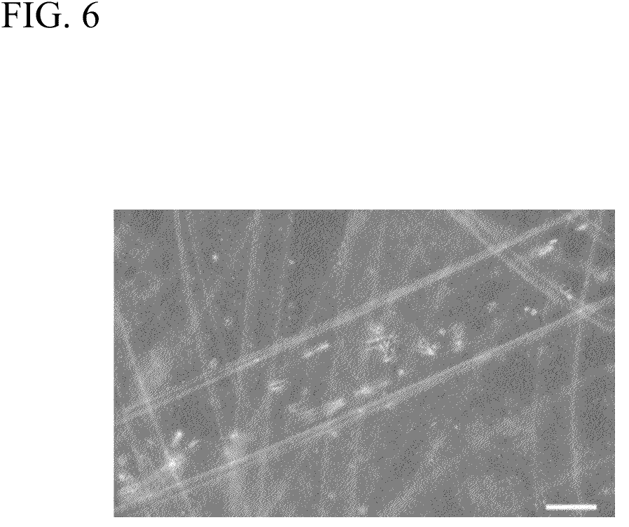

FIG. 4 is a schematic diagram showing a structure of the scaffold for culturing cells according to the second embodiment. FIG. 5 is a schematic diagram showing a cross section of the scaffold for culturing cells according to the second embodiment. In the embodiment shown in FIGS. 4 and 5, a scaffold 20 has a hydrogel 22 in a form of a continuously extending tube. A plasma-derived or platelet-derived component, or a fibrin-containing material 24 is provided on the inner surface of the hydrogel 22. In this case, adherent cells 28 are cultured while adhering to a component 24 on the inner surface of the hydrogel 22. That is, the adherent cells 28 are cultured inside the scaffold in a form of the tube (see FIG. 6). FIG. 6 shows an enlarged portion of a cell culture obtained by adhering mesenchymal stem cells (MSCs) to the inside of a scaffold for culturing cells, and culturing the MSCs. Further, by continuing to culture the cells, it is possible to culture the cells until the inner portion of the scaffold in a form of a tube becomes dense.

The type of adherent cells is not particularly limited. The adherent cells may be various stem cells having multipotency, human embryonic stem (ES) cells or human induced pluripotent stem (iPS) cells, having pluripotency, stem cells having differentiation unity, or the like. Examples of the various stem cells having multipotency include iPS cells, ES cells, mesenchymal stem cells, and neural stem cells. Examples of the stem cells having differentiation unity include hepatic stem cells, germline stem cells, respiratory progenitor, and gastrointestinal progenitor. The adherent cells may also be various types of differentiated cells, for example, muscle cells such as skeletal muscle cells and myocardial cells, nerve cells such as cerebral cortical cells, fibroblasts, epithelial cells, endothelial cells, adipocytes, osteoblasts, macrophages, dendritic cells, liver cells, pancreatic β cells, keratinocytes, kidney cells, tubular cells, and the like.

The hydrogel is obtained by turning a hydrogel precursor in a liquid state into a gel. It is sufficient that the hydrogel has a strength enough to function as a scaffold for adherent cells, and preferably, the hydrogel has sufficient permeability to a cell culture medium component. In the specific example, the hydrogel may be a gel using an alginate gel as the main component. In this case, the hydrogel precursor may be a solution using an alginate solution as the main component.

The hydrogel may contain another material mixed in an alginate gel. For example, gelatin or collagen may be mixed in an alginate gel. The gelatin or collagen may be crosslinked by a crosslinking agent. The crosslinking agent is not particularly limited, and may be, for example, Genipin.

The alginate gel can be formed by crosslinking an alginate solution with divalent metal ions. The alginate solution may be, for example, sodium alginate, potassium alginate or ammonium alginate, or a combination thereof. The alginate solution is easily crosslinked with divalent metal ions in a short time at room temperature or at the vicinity of room temperature, and easily turns into an alginate gel. Further, the alginate gel is not cytotoxic. Accordingly, the hydrogel constituting a scaffold for culturing cells preferably contains an alginate gel as the main component.

Alginic acids may be a natural extract or a chemically modified one. Chemically modified alginic acid includes, for example, methacrylate-modified alginic acid. Further, the hydrogel may be a mixed system of the above-described alginate, with agar, agarose, polyethylene glycol (PEG), polylactic acid (PLA), nano-cellulose, or the like. The weight of the alginate to the weight of a solvent of the alginate solution is, for example, 0.1 to 10.0 wt%, preferably 0.25 to 7.0 wt%, and more preferably 0.5 to 5.0 wt%.

Examples of the divalent metal ions used to obtain an alginate gel include calcium ions, magnesium ions, barium ions, strontium ions, zinc ions, and ferrous ions. Preferably, the divalent metal ions are calcium ions or barium ions.

The divalent metal ions are preferably given to alginic acid by the form of a solution. A solution containing divalent metal ions includes, for example, a solution containing calcium ions. Examples of such a solution include solutions such as a calcium chloride aqueous solution, a calcium carbonate aqueous solution, and a calcium gluconate aqueous solution. Such a solution may be preferably a calcium chloride aqueous solution or a barium chloride aqueous solution.

The concentration of divalent metal ions in a solution containing the divalent metal ions is, for example, 1 mM to 1 M, preferably 20 to 500 mM, and more preferably 100 mM.

A raw material of the alginate gel may be preferably sodium alginate. In this case, from the viewpoint of the strength as a scaffold and the permeability of a medium component, the M/G ratio of the sodium alginate is preferably 0.4 to 1.8, and more preferably 0.1 to 0.4. The M/G ratio is defined by the constituent ratio of D-mannuronic acid to L-guluronic acid of alginic acids.

The above-described plasma-derived or platelet-derived component may include a human platelet lysate (hPL)-derived component. Such a component may be, for example, an insoluble component in a human platelet lysate, or a fibrin-containing material. The fibrin-containing material may contain a plasma-derived, platelet-derived, or human platelet lysate-derived insoluble component.

Further, the “fibrin-containing material” may be fibrin itself, may contain fibrin as the main component, or may contain a component other than the fibrin. Examples of the component other than the fibrin include proteins such as albumin and fibronectin. As an example, the fibrin-containing material may contain both of fibrin and albumin. In addition, a medium in a liquid state containing a human platelet lysate (hPL) may contain, for example, a plasma or hPL-derived component, fibrin, fibrinogen, or a mixture thereof. Fibrinogen is converted to fibrin by the action of thrombin. Fibrin is an insoluble component, and can be allowed to adhere onto the inner surface or outer surface of a hydrogel. Further, it can be expected that fibrin is easily adhered onto the inner surface or outer surface of a hydrogel by a protein such as albumin or fibronectin.

A plasma-derived or platelet-derived component or a fibrin-containing material may be adhered to a hydrogel by any method. For example, by bringing a suspension containing a plasma-derived or platelet-derived component or fibrinogen into contact with a hydrogel, the plasma-derived or platelet-derived component or a fibrin-containing material can be adhered to the hydrogel.

The plasma-derived or platelet-derived component, fibrin, or fibrinogen is contained in a medium in a liquid state containing, for example, a platelet lysate. Accordingly, a scaffold can be formed by bringing a suspension that contains a medium in a liquid state containing a platelet lysate, for example, a human platelet lysate (hPL) into contact with a hydrogel. The suspension may be a cell suspension containing cells to be cultured by adhering the cells to the scaffold. Alternatively, fibrin can be adhered to a hydrogel by the action of thrombin while a solution containing fibrinogen is in contact with the hydrogel. In addition, it should be noted that if fibrin can be precipitated, the use of thrombin is not essential.

Preferably, the inner surface or outer surface of a hydrogel is coated with a plasma-derived or platelet-derived component, or a fibrin-containing material.

In a case where the scaffold has a string shape or a tube shape, the length of the hydrogel may be, for example, preferably 5 cm or more, and more preferably 10 cm or more, and furthermore preferably 20 cm or more. As the length of the hydrogel is longer, the more cells can be cultured.

In a case where the scaffold has a form of a continuously extending string as shown in FIGS. 1 and 2, the outer diameter of the scaffold (reference sign R1 in FIG. 2) is not particularly limited. From the viewpoint of the adhesiveness of adherent cells, the outer diameter of the scaffold may be, for example, in the range of 10 µm to 5000 µm, preferably in the range of 40 µm to 2000 µm, and more preferably in the range of 80 µm to 1000 µm. The outer diameter may be defined by an average value of the outer diameters measured at multiple positions, for example, at 10 positions.

In a case where the scaffold has a form of a continuously extending tube as shown in FIGS. 4 and 5, the outer diameter of the scaffold (reference sign R2 in FIG. 5) is not particularly limited. The outer diameter of the scaffold may be, for example, in the range of 10 µm to 4000 µm, preferably in the range of 40 µm to 1000 µm, and more preferably in the range of 80 µm to 500 µm. The outer diameter may be defined by an average value of the outer diameters measured at multiple positions, for example, at 10 positions.

The thickness of the hydrogel constituting a scaffold (difference between R2 and R3 in FIG. 5) is preferably substantially uniform. The expression “substantially uniform” means that the difference (thickness) between the outer diameter and the inner diameter, measured at multiple positions, for example, at 10 positions is within the range of ± 10% from the average value. In addition, the above-described inner diameter, outer diameter, and thickness can be measured with, for example, a phase contrast light microscope.

In a case where the hydrogel constituting a scaffold is in a form of a tube, both ends in the extending directions of the hydrogel are preferably closed with the hydrogel. In this way, the cells inside the hydrogel can be prevented from leaking out of the hydrogel.

From the viewpoint of protecting the cells, the hydrogel in a form of a tube preferably has a mechanical strength higher than the strength of a substrate arranged inside the hydrogel. As for the mechanical strength of the hydrogel, the tensile strength and the load strength can be measured by a method using a tensile testing machine in water, or the like, in accordance with a method well known to those skilled in the art.

The inside of the hydrogel having the tube shape may contain a cell suspension. The cell suspension may contain the above-described cells to be cultured, a plasma-derived or platelet-derived component or a component being the source of a fibrin-containing material which are to be allowed to adhere to the hydrogel, and a substrate. The cell suspension may be the one used for the adhesion of the above-described plasma-derived or platelet-derived component or the fibrin-containing material. The substrate may contain a group selected from the group consisting of, for example, an extracellular matrix, a chitosan gel, a collagen solution, Matrigel, a collagen gel, gelatin, an alginate solution, an alginate gel, a peptide gel, laminin, agarose, nano-cellulose, methyl cellulose, hyaluronic acid, a proteoglycan, elastin, pullulan, dextran, pectin, gellan gum, xanthane gum, guar gum, carrageenan, and glucomannan, or a mixture thereof. Further, the cell suspension may contain a medium, a culture supernatant, a buffer solution, a human platelet lysate, a platelet rich plasma (PRP), serum, or a mixture thereof. Preferably, the cell suspension contains a human platelet lysate.

The substrate may contain various kinds of growth factors suitable for culturing cells, maintaining or proliferating cells, developing cell function, or the like, for example, an epidermal growth factor (EGF), a platelet-derived growth factor (PDGF), a transforming growth factor (TGF), an insulin-like growth factor (IGF), a fibroblast growth factor (FGF), a nerve growth factor (NGF), a vascular endothelial growth factor (VEGF), a hepatocyte growth factor (HGF)and the like.

### Method for Producing Scaffold 1

In a case where a scaffold for culturing cells has a form of a continuously extending string as shown in FIGS. 1 and 2, the scaffold can be produced, for example, as follows. First, a hydrogel precursor is turned into a gel to form a hydrogel. The hydrogel precursor may be any precursor capable of turning into a gel. Such a precursor includes, for example, a solution using an alginate solution as the main component. The alginate solution may be, for example, a sodium alginate aqueous solution. If necessary, a solution such as a gelatin solution or a collagen solution may be mixed in the alginate solution.

Next, after filling a syringe with the hydrogel precursor, the hydrogel precursor is ejected from an ejection port of the syringe into a solution containing a gelling agent at a predetermined rate. In a case where the hydrogel precursor is an alginate solution, the solution containing a gelling agent may be a solution containing the above-described divalent cations. The solution containing divalent cations may be, for example, a solution obtained by dissolving calcium chloride or barium chloride in a desired medium in a liquid state. Further, in a case where the hydrogel precursor contains a gelatin solution or a collagen solution, the solution containing a gelling agent may contain a crosslinking agent such as Genipin.

The hydrogel precursor ejected into the solution containing a gelling agent is gelled to form a hydrogel. By continuously ejecting the hydrogel precursor from an ejection port of the syringe, the hydrogel is formed in a form of a continuously extending string.

Next, the hydrogel in a form of a string, which is obtained as described above, is immersed in a suspension containing a plasma-derived or platelet-derived component, fibrin or fibrinogen, or a mixture thereof. The components of this suspension are as described above. The suspension may be, for example, a medium in a liquid state containing a plasma or hPL-derived insoluble component, and more preferably a medium containing a human platelet lysate.

With the above processes, a plasma-derived or platelet-derived component, and/or a fibrin-containing material converted from fibrinogen adhere onto the outer surface of a hydrogel in a form of a string. In this way, the above-described scaffold is formed. In addition, if the hydrogel precursor is intermittently ejected from the ejection port of a syringe, a hydrogel in a form of a large number of spheres is formed. If the fibrin-containing material is adhered to the hydrogel in a form of a large number of spheres as described above, a scaffold is formed in a sphere shape.

In a case where adherent cells are cultured by using such a scaffold, the scaffold may be put into a desired medium in a liquid state, and further adherent cells may be seeded on it. In this way, the adherent cells are cultured while adhering to a plasma-derived or platelet-derived component or a fibrin-containing material on the outer surface of a hydrogel.

### Method for Producing Scaffold 2

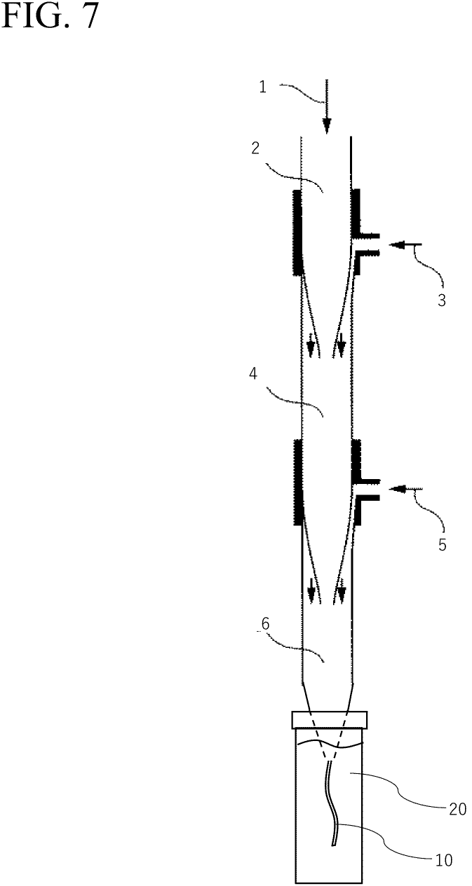

In a case where the scaffold for culturing cells has a form of a continuously extending tube as shown in FIGS. 4 and 5, the scaffold can be produced, for example, as follows. FIG. 7 is a schematic diagram of an apparatus used for forming a scaffold in a form of a tube.

First, while flowing a suspension 1 containing a plasma-derived or platelet-derived component, fibrin or fibrinogen, or a mixture thereof, a hydrogel precursor 3 flows around the suspension 1. The suspension 1 is a cell suspension which contains cells to be cultured and a substrate containing fibrin and/or fibrinogen. The materials of the substrate are as described above. Preferably, the substrate contains an extracellular matrix and/or a human platelet lysate. In this case, the cell suspension contains a plasma or hPL-derived component, fibrin or fibrinogen, or a mixture thereof. The materials of the hydrogel precursor 3 are as described above.

Preferably, the suspension 1 is flowed as a laminar flow. The laminar flow is formed in a first introduction pipe 2. The hydrogel precursor 3 is flowed so as to cover the outer periphery of the flow of the suspension 1. That is, the hydrogel precursor 3 flows coaxially with the suspension 1 in the same direction as that of the suspension 1. Preferably, the hydrogel precursor 3 is flowed as a laminar flow. In this way, a flow of the hydrogel precursor 3 surrounding the flow of the cell suspension 1 is formed at a second introduction pipe 4.

The hydrogel precursor is gelled to form a hydrogel in a form of a tube shape covering the suspension 1. This can be achieved by bringing a solution 5 containing a gelling agent with which the hydrogel precursor 3 turns into a gel, into contact with the outer periphery of the flow of the hydrogel precursor 3. In the embodiment shown in FIG. 7, the solution 5 surrounds the periphery of the hydrogel precursor (second laminar flow) 3 at a third introduction pipe 6. That is, the solution 5 flows coaxially with the suspension 1 and the hydrogel precursor 3 in the same direction as those of the suspension 1 and the hydrogel precursor 3.

The cell suspension 1, the hydrogel precursor 3, and the solution 5 containing a gelling agent may start flowing in any order at the time of starting, and may stop flowing in any order at the time of stopping. However, from the viewpoint of confining the cells without leakage, the cell suspension 1 preferably starts last at the time of starting the production, and desirably stops first at the time of stopping the production. Each flow rate of the cell suspension 1, the hydrogel precursor 3, and the solution 5 containing a gelling agent is not particularly limited as long as a scaffold is formed.

The cell suspension 1, the hydrogel precursor 3, and the solution 5 containing a gelling agent flow out from a third introduction pipe 6, and are immersed in, for example, a liquid such as a saline solution or a medium in a liquid state, or in a suspension. The hydrogel precursor 3 flows out from a third introduction pipe 6 while turning into a gel by the application of the gelling agent. In this way, a hydrogel which covers the suspension is formed in a form of a tube. An insoluble component formed inside the hydrogel in a form of a tube, that is, a plasma-derived or platelet-derived component and/or a fibrin-containing material adhere onto the inner surface of the hydrogel in a form of a tube with the lapse of time.

With the above processes, a plasma-derived or platelet-derived component and/or a fibrin-containing material adhere on the inner surface of the hydrogel in a form of a tube. In this way, the above-described scaffold is formed.

In the embodiment shown in FIG. 7, the scaffold is formed by forming a flow of a cell suspension 1, a flow of a hydrogel precursor 3, and a flow of a solution 5 containing a gelling agent, and by flowing them out from the third introduction pipe 6. Alternatively, the scaffold with a similar structure can also be formed by forming a flow of a cell suspension 1, forming a flow of a hydrogel precursor 3 to cover the outer periphery of the first laminar flow, and ejecting these flows into a container that stores a solution 5 containing a gelling agent. In this case, if the flow of a cell suspension 1 and the flow of a hydrogel precursor 3 are intermittently ejected, the hydrogel is formed in a form of a large number of spherical shells. As described above, a plasma-derived or platelet-derived component and/or a fibrin-containing material adhere onto the inner surface of the hydrogel in a form of spherical shells. In this case, the scaffold which encases cells can be formed in a form of a spherical shell.

In a case where adherent cells are cultured by using such a scaffold, the adherent cells to be cultured may be contained inside the hydrogel. In this way, the adherent cells are cultured while adhering to a plasma-derived or platelet-derived component and/or a fibrin-containing material on the inner surface of the hydrogel.

The hydrogel constituting a scaffold for culturing cells is preferably an alginate gel. Such an alginate gel has the advantage that it can easily and immediately turns into a gel from an alginate solution by a solution containing divalent cations at room temperature or at the vicinity of room temperature. Further, the alginate gel has the advantage of being weakly cytotoxic.

However, because of being non-adhesive to cells, the alginate gel is not necessarily suitable as a scaffold for adherent cells. The inventors of the present application have found a scaffold with which adherent cells are easily cultured while adhering the adherent cells to the scaffold by adhering a plasma-derived or platelet-derived component and/or a fibrin-containing material on the outer surface or inner surface of a hydrogel, particularly, an alginate gel.

Further, the inventors of the present application have found that a medium in a liquid state containing a human platelet lysate (suspension) is suitably utilizable in order to allow effectively a plasma-derived or platelet-derived component and/or a fibrin-containing material to adhere/coat on the hydrogel, particularly an alginate gel, in a form of a string shape, a tube shape, a sphere shape or a spherical shell shape as described above. The method for the adhesion or coating is not limited to the method described above as long as the plasma-derived or platelet-derived component and/or the fibrin-containing material can be allowed to adhere onto the outer surface or inner surface of the hydrogel by the method.

Further, in order to make it easier to allow a plasma-derived or platelet-derived component and/or a fibrin-containing material to adhere to a hydrogel, a suspension is generated by mixing the plasma-derived or platelet-derived component or a solution containing fibrinogen with a medium, and then the generated suspension may be left to stand at room temperature (for example, temperature of 22 to 37° C.) for 10 to 120 minutes. After the standing, the above suspension may be brought into contact with a hydrogel. Further, in a case where the scaffold for culturing cells is in a form of a continuously extending tube as shown in FIGS. 4 and 5, it is preferable that a suspension is generated by mixing a solution containing a plasma-derived or platelet-derived component or fibrinogen with a medium, and then the generated suspension is left to stand at room temperature (for example, temperature of 22 to 37° C.) for 10 to 120 minutes before introducing the cells to be cultured into a suspension 1. Using the cell suspension 1 thus obtained, a scaffold in a form of a tube may be produced by using an apparatus shown in FIG. 7 as described above.

In addition, the inventors of the present application have found that adherent cells can be easily cultured while allowing the adherent cells to adhere more firmly by mixing a collagen gel or a gelatin gel into an alginate gel as necessary. Since a collagen gel or a gelatin gel is more highly adhesive to cells as compared with an alginate gel, the disadvantage of an alginate gel, which is low adhesive to cells, can be compensated. In this case, the ratio of the collagen gel or gelatin gel to the alginate gel may be, for example, 0.1 to 20% by mass, and preferably 1.0 to 20% by mass.

### Example 1

### Production of Scaffold

Examples will be described in detail. First, as the reagents, the following ones were prepared.
Sodium alginate (“I-3G” manufactured by KIMICA Corporation)
Gelatin (“beMatrix gelatin LS-H” manufactured by Nitta Gelatin Inc.)
Dulbecco’s modified Eagle’s medium: DMEM (“D6429” manufactured by Sigma-Aldrich Co. LLC.)
Genipin (“Product code: 078-03021” manufactured by FUJIFILM Wako Pure Chemical Corporation)
Medium for MSC (Mesenchymal Stem Cell Growth Medium 2 (Ready-to-use): C-28009)
hPL solution (AventaCell, UltraGRO-PURE: HPCHXCRL50))

First, a solution obtained by adding the above gelatin to a saline solution was autoclaved to prepare a gelatin solution. The concentration of the gelatin to the saline solution was a concentration equivalent to 20% by volume at a temperature of 37° C.

Further, an alginate solution was prepared by adding the above sodium alginate to a saline solution, and stirring them. The concentration of the sodium alginate to the saline solution was 1.96% by mass.

A mixture was prepared by mixing the above-described gelatin solution with the above-described alginate solution so as to have a volume ratio of 1 : 1 at a temperature of 37° C.

Further, calcium chloride and the above Genipin were dissolved in Dulbecco’s modified Eagle’s medium (DMEM). The concentration of the Genipin to the medium (DMEM) was 1 mM at a temperature of 37° C. In addition, the concentration of the calcium chloride to the medium (DMEM) was 100 mM at a temperature of 37° C. The expression “M” means molar concentration (mol/L) (hereinafter, the same applies). In this way, a medium in a liquid state containing calcium chloride was prepared.

Next, a glass syringe was filled with the mixture of the gelatin solution with the alginate solution. This mixture was ejected through an injection needle attached to the syringe into a medium in a liquid state containing the above calcium chloride.

The alginate solution in the mixture is crosslinked by calcium ions in the medium in a liquid state and turns into a gel. In addition, the gelatin in the mixture is crosslinked by Genipin in the medium in a liquid state. In this way, a hydrogel (scaffold) with a string shape is formed in the medium in a liquid state (see also FIG. 1).

After the mixture was ejected from the syringe, the liquid medium containing the hydrogel in a form of a string shape was left to stand for a desired time while being maintained at 37° C.

After the standing, the hydrogel with the string shape was washed, and then the hydrogel with the string shape was transferred to a mixture of the above hPL solution and the above medium for MSC. The medium storing the hydrogel was left to stand for a desired time while being maintained at 37° C. In addition, the concentration of the hPL solution to the medium for MSC was such that the volume ratio was 1 : 1 at a temperature of 37° C. The hPL solution contains a plasma-derived or platelet-derived component, fibrinogen or fibrin, or a mixture thereof.

With the above procedures, a scaffold to which a plasma-derived or platelet-derived component and/or a fibrin-containing material have adhered is formed on the outer surface of a hydrogel in a form of a string. The plasma-derived or platelet-derived component and/or the fibrin-containing material are derived from the components in the hPL solution. In addition, the diameter of the scaffold in a form of a string was around 420 µm.

### Culture of Cells

A method for culturing cells by using the scaffold produced in Example 1 will be described. First, the following cells, materials, and reagents were prepared.
Scaffold in a form of a string produced as described above
Human mesenchymal stem cells from bone marrow: hMSC-BM (“C-12974” manufactured by Promo Cell)
Medium (Mesenchymal Stem Cell Growth Medium 2 (Ready-to-use): C-28009)

The above-described scaffold with the string shape was transferred to the above medium for MSC. The human mesenchymal stem cells from bone marrow dispersed in this medium were seeded. The number of the seeded cells was 4.0×10⁵ cells. Further, the container storing the medium was non-adhesive to cells.

Under these conditions, cells were cultured for the number of days shown in Table 1. In addition, the entire amount of the medium was replaced every 2 days. The medium to be used after the replacement is the same as the medium used before the replacement. In a case where cells not adhering to the scaffold were present, such cells were recovered by centrifuging the supernatant of the medium. The recovered cells were seeded again in a medium after medium replacement.

### Recovery of Cells

A method for recovering the cells cultured by using the above-described scaffold will be described. First, a hydrogel (scaffold) in a form of a string, to which cells had adhered, was taken out from the medium, and put into a recovery liquid obtained by mixing a EDTA/PBS solution with an enzyme solution for cell dispersion (Tryple). While maintaining the scaffold in the recovery liquid, the incubation was performed at 37° C. for 5 minutes to dissolve the scaffold and disperse the cells.

Next, the incubation was further performed at 37° C. for more 3 minutes, and when the shape of the scaffold became invisible, the recovery liquid was centrifuged for 5 minutes. After that, the supernatant of the recovery liquid was discarded, the remaining portion was suspended in a medium, the recovered cells were stained with trypan blue, and the number of living cells was counted.

### Example 2

The scaffold and production method thereof according to Example 2 were the same as those in Example 1 except for the following points. In Example 2, the above hPL solution (AventaCell, UltraGRO-PURE: HPCHXCRL50) was introduced into a medium for MSC during culturing cells. The concentration of the hPL solution to the medium for MSC was equivalent to 10% by volume at a temperature of 37° C. Other matters were similar to those in Example 1.

### Reference Example 1

The scaffold according to Reference Example 1 was substantially similar to Example 1 except that a plasma-derived or platelet-derived component and/or a fibrin-containing material were not adhered onto the outer surface of the hydrogel with the string shape. Accordingly, in Reference Example 1, after preparing the hydrogel with the string shape in a similar manner to Example 1, the hydrogel with the string shape was not put into the hPL solution. Other matters were similar to those in Example 1.

### Reference Example 2

The scaffold and production method thereof according to Reference Example 2 were the same as those in Reference Example 1 except for the following points. In Reference Example 2, the above hPL solution (AventaCell, UltraGRO-PURE: HPCHXCRL50) was introduced into the medium for MSC during culturing cells. The concentration of the hPL solution to the medium for MSC was equivalent to 10% by volume at a temperature of 37° C. Other matters were similar to those in Reference Example 1.

The results of culturing cells for Examples 1 and 2, and Reference Examples 1 and 2 are shown in Table 1 below.

TABLE 1

Cell culture on the outer surface of the hydrogel

| | hPL treatment on the outer surface of the hydrogel | Introdution of hPL into the medium | Number of seeded cells (cells) | Number of recovered cells (cells) | Proliferation rate (times) | Culture days (days) | Doubling time (time) |
|---|---|---|---|---|---|---|---|
| Example 1 | Yes | No | 4.0×10⁵ | 4.0×10⁶ | 10 | 4 | 28.9 |
| Example 2 | Yes | Yes | 4.0×10⁵ | 3.9×10⁶ | 9.75 | 4 | 29.2 |
| Reference Example 1 | No | No | 4.0×10⁵ | 1.3×10⁶ | 3.25 | 4 | 56.5 |
| Reference Example 2 | No | Yes | 4.0×10⁵ | 3.6×10⁶ | 9 | 4 | 30.3 |

The proliferation rates of the cells in Examples 1 and 2, and Reference Example 1 was 9 times or more, and were each larger than that in Reference Example 1. Further, the proliferation rates of the cells in Examples 1 and 2 were each slightly larger than that in Reference Example 2. Similarly, the doubling times of cell culture in Examples 1 and 2 were each improved rather than the doubling times of Reference Examples 1 and 2. This indicates that a scaffold obtained by adhering a plasma-derived or platelet-derived component and/or a fibrin-containing material onto the outer surface of a hydrogel in a form of a string is more suitable for culturing cells.

In addition, with a comparison between Examples 1 and 2 or a comparison between Example 1 and Reference Example 2, it can be understood that even if a human platelet lysate (hPL) was not introduced into a medium during culturing cells, the proliferation rate of cells can be sufficiently increased by using a scaffold obtained by allowing a plasma-derived or platelet-derived component and/or a fibrin-containing material to adhere onto the outer surface of a hydrogel in a form of a string. That is, if a scaffold obtained by allowing a plasma-derived or platelet-derived component and/or a fibrin-containing material to adhere onto the outer surface of a hydrogel in a form of a string is used, there is no need to introduce a human platelet lysate into the medium during culturing cells. As a result, it becomes possible to reduce the amount of the human platelet lysate to be used, which is relatively expensive.

### Example 3

Example 3 corresponds to a subculture of the cells recovered from Example 1. Specifically, except for the following description, a scaffold was produced in a similar manner to Example 1, and cells were cultured also in a similar manner to Example 1. However, the cells seeded in Example 3 are the cells recovered in Example 1. The number of the cells seeded in Example 3 was 4.0×10⁵ cells.

### Example 4

Example 4 corresponds to a subculture of the cells recovered in Example 2. Specifically, except for the following description, a scaffold was produced in a similar manner to Example 2, and cells were cultured also in a similar manner to Example 2. That is, in Example 4, a human platelet lysate is introduced into a medium for MSC during culturing cells. However, the cells seeded in Example 4 are the cells recovered in Example 2. The number of the cells seeded in Example 4 was 4.0×10⁵ cells.

### Reference Example 3

Reference Example 3 corresponds to a subculture of the cells recovered in Reference Example 1. Specifically, except for the following description, a scaffold was produced in a similar manner to Reference Example 1, and cells were cultured also in a similar manner to Reference Example 1. However, the cells seeded in Reference Example 3 are the cells recovered in Reference Example 1. The number of the cells seeded in Reference Example 3 was 4.0×10⁵ cells.

### Reference Example 4

Reference Example 4 corresponds to a subculture of the cells recovered in Reference Example 2. Specifically, except for the following description, a scaffold was produced in a similar manner to Reference Example 2, and cells were cultured also in a similar manner to Reference Example 2. That is, in Reference Example 4, a human platelet lysate is introduced into a medium for MSC during culturing cells. However, the cells seeded in Reference Example 4 are the cells recovered in Reference Example 2. The number of the cells seeded in Reference Example 4 was 4.0×10⁵ cells.

The results of culturing cells for Examples 3 and 4, and Reference Examples 3 and 4 are shown in Table 2 below.

TABLE 2

Cell culture on the outer surface of the hydrogel (SUBCULTURE)

| | hPL treatment on the outer surface of the hydrogel | Introdution of hPL into the medium | Number of seeded cells (cells) | Number of recovered cells (cells) | Proliferation rate (times) | Culture days (days) | Doubling time (time) |
|---|---|---|---|---|---|---|---|
| Example 3 | Yes | No | 4.0×10⁵ | 1.7×10⁶ | 4.25 | 5 | 57.5 |
| Example 4 | Yes | Yes | 4.0×10⁵ | 1.3×10⁶ | 3.25 | 5 | 70.6 |
| Reference Example 3 | No | No | 4.0×10⁵ | 2.1×10⁵ | 0.525 | 5 | - |
| Reference Example 4 | No | Yes | 4.0×10⁵ | 3.8×10⁵ | 0.95 | 5 | - |

In Examples 3 and 4, in a case where the scaffold of the hydrogel to which a plasma-derived or platelet-derived component and/or a fibrin-containing material is adhered was used, the proliferation rate was maintained to be 3 times or more even in the subculture of cells. In Reference Examples 3 and 4, the number of recovered cells was reduced. This indicates that the scaffolds of Examples 3 and 4 are more suitable for culturing cells.

### Example 5

Example 5 corresponds to a re-subculture of the cells recovered in Example 3. Specifically, except for the following description, a scaffold was produced in a similar manner to Example 1, and cells were cultured also in a similar manner to Example 1. However, the cells seeded in Example 5 are the cells recovered in Example 3. The number of the cells seeded in Example 5 was 4.0×10⁵ cells.

In Example 5, the number of the cells recovered after the cells were cultured for 5 days was 1.6×10⁶ cells, and the proliferation rate was maintained to be around 4 times. As described above, in a case where a scaffold of a hydrogel to which a plasma-derived or platelet-derived component and/or a fibrin-containing material had adhered was used, the high proliferation rate was maintained even in the re-subculture of cells.

It has been found that mesenchymal stem cells are adherent cells, and easily adhere to the scaffolds of Examples 1 to 5. In this way, by using a scaffold obtained by allowing a plasma-derived or platelet-derived component and/or a fibrin-containing material to adhere onto the outer surface of a hydrogel, the effect of culturing adherent cells can be improved.

Further, as described above, by making the scaffold into a string shape, it becomes easy to handle the scaffold during culture, and further three-dimensional culture in a medium in a liquid state becomes easy. However, from the viewpoint of the effect of culturing adherent cells, the scaffold is not limited to the string shape, and may be, for example, a spherical shape.

### Example 6

### Production of Scaffold

Example 6 will be described in detail. The scaffold produced in Example 6 is a scaffold in a form of a tube as shown in FIGS. 4 and 5. First, as the reagents, the following ones were prepared.
Sodium alginate (“I-3G” manufactured by KIMICA Corporation)
Human mesenchymal stem cells from bone marrow: hMSC-BM (“C-12974” manufactured by Promo Cell)
Dulbecco’s modified Eagle’s medium: DMEM (“D6046” manufactured by Sigma-Aldrich Co. LLC.)
Human platelet lysate: STEMCELL TECHNOLOGIES, 06960

First, a cell suspension, a sodium alginate solution, and a calcium chloride aqueous solution were prepared. The sodium alginate solution was generated by adding the above sodium alginate to a saline solution, and stirring them. The concentration of the sodium alginate to the saline solution was 1.44% by mass.

The cell suspension contains the above human mesenchymal stem cells from bone marrow, a mixture of the above Dulbecco’s modified Eagle’s medium (DMEM) and the above fetal bovine serum (FBS), and the above human platelet lysate. Specifically, the DMEM and the FBS were mixed so as to have a volume ratio of 9 : 1 at a temperature of 37° C. Further, the above human platelet lysate was mixed so as to have a volume ratio of the human platelet lysate to the mixture of the DMEM and the FBS of 7 : 3 at a temperature of 37° C. Then, the resultant minute was left to stand at room temperature for 10 to 120 minutes. Next, a cell suspension was generated by introducing the human mesenchymal stem cells from bone marrow into the mixture of the DMEM, the FBS, and the human platelet lysate. The density of the human mesenchymal stem cells from bone marrow in the suspension was 2×10⁵ cells/mL. The FBS contains abundant albumin.

By using these materials, a scaffold of a hydrogel with the tube shape covering the cell suspension was prepared in accordance with the method for preparing a scaffold shown in FIG. 7 and the description accompanying FIG. 7. That is, a flow of the cell suspension, a flow of a sodium alginate solution around the flow of the cell suspension, and a flow of a calcium chloride aqueous solution around the flow of the sodium alginate solution were formed, and these flows were ejected into a saline solution (see also FIG. 7). The sodium alginate solution was crosslinked by being brought into contact with a calcium chloride aqueous solution, and an alginate gel was formed. In this way, the hydrogel in a form of an elongated tube encasing the cell suspension was generated in the saline solution. The number of cells in the hydrogel in a form of the tube shape was approximately 2×10⁴ cells. The diameter of the cross section of the generated hydrogel with the tube shape was 400 to 500 µm.

The hydrogel having the elongated tube encasing the cell suspension was left to stand for a desired time in a saline solution. Next, the hydrogel having the cylindrical shape encasing the cell suspension was transferred to a liquid medium, and cells were cultured in the hydrogel with the tube shape. It is considered that in these processes, a human platelet lysate-derived insoluble component, specifically, a plasma-derived or platelet-derived component and/or a fibrin-containing material is adhered on the inner surface of the hydrogel. In this way, the scaffold for culturing cells was produced.

The human mesenchymal stem cells from bone marrow were cultured inside of the scaffold in a liquid medium at 37° C. for the number of days shown in Table 3. This liquid medium is the above-described Dulbecco’s modified Eagle’s medium: DMEM (“D6046” manufactured by Sigma-Aldrich Co. LLC.). In addition, the liquid medium was replaced every 2 days. The medium to be used after the replacement is the same as the medium used before the replacement.

### Recovery of Cells

A method for recovering the cells cultured by using the above-described scaffold will be described. First, the hydrogel (scaffold) in a form of a tube enclosing cells was removed by an EDTA/PBS solution, and the cells were recovered by centrifugation. Thus, the recovered cells were stained with trypan blue, and the number of living cells was counted. Further, the cells were recovered by the present method on the fourth day from the start of culturing cells, and the recovered cells were subcultured. In the subculture, the recovered cells were cultured in the scaffold having the tube shape in a similar manner to the above method.

### Example 7

The method for producing a scaffold and method for culturing cells according to Example 7 were similar to those in Example 6, except for the following description. In Example 7, during the preparation of a cell suspension, a human platelet lysate was mixed with a mixture of DMEM and FBS so that a volume ratio of the human platelet lysate to the mixture of DMEM and FBS becomes 4 : 6 at a temperature of 37° C. The other steps were the same as those in Example 6.

### Example 8

The method for producing a scaffold and method for culturing cells according to Example 8 were similar to those in Example 6, except for the following description. In Example 8, a human platelet lysate was used without using a mixture of DMEM and FBS during the preparation of a cell suspension. The other steps were the same as those in Example 6.

### Reference Example 5

The method for producing a scaffold and method for culturing cells according to Reference Example 5 were similar to those in Example 6, except for the following description. In Reference Example 5, a human platelet lysate was not mixed with a mixture of DMEM and FBS during the preparation of a cell suspension. That is, the cell suspension was formed by introducing human mesenchymal stem cells from bone marrow into a mixture of DMEM and FBS. The other steps were the same as those in Example 6.

The results of culturing cells for Examples 6, 7, and 8, and Reference Example 5 are shown in Table 3 below.

TABLE 3

Cell culture inside of the hydrogel

| | Introduction of hPL to the cell suspension into the hydrogel | Introdution of hPL into the medium | Number of seeded cells (cells) | Proliferat ion rate (times) | Culture days (days) | Doubling time (time) |
|---|---|---|---|---|---|---|
| Example 6 | 30% | No | 2.0×10⁴ | 2.2 | 9 | 189.9 |
| Example 7 | 60% | No | 2.0×10⁴ | 6.2 | 9 | 82.1 |
| Example 8 | 100% | No | 2.0×10⁴ | 9.2 | 9 | 67.5 |
| Reference Example 5 | No | No | 2.0×10⁴ | 0.4 | 9 | - |

The proliferation rates of the cells in Examples 6 to 8 were each higher than that in Reference Example 5. Therefore, it has been found that the proliferation rate of cells is higher when a human platelet lysate was mixed with a cell suspension. In other words, it has been found that the scaffold obtained by adhering the plasma-derived or platelet-derived component or the fibrin-containing material to the inner surface of the hydrogel in a form of the tube shape is more suitable for culturing cells.

Further, referring to Examples 6 to 8, it can be understood that the higher the concentration of a human platelet lysate in a cell suspension is when a scaffold is produced, the higher the proliferation rate of cells is and the shorter the doubling time is.

### Example 9

The method for producing a scaffold and method for culturing cells according to Example 9 were similar to those in Example 6, except for the following description. In Example 9, during the preparation of the cell suspension, a human platelet lysate was mixed with a mixture of DMEM and FBS so that a volume ratio of the human platelet lysate to the mixture of DMEM and FBS becomes 9 : 1 at a temperature of 37° C. Further, the liquid medium to be used during culturing cells was set to be one obtained by mixing a hPL solution (AventaCell, UltraGRO-PURE: HPCHXCRL50) with the above-described mixture of DMEM and FBS. The hPL solution was mixed with the mixture of DMEM and FBS so that a volume ratio of the hPL solution to the mixture of DMEM and FBS becomes 9 : 1 at a temperature of 37° C. The other steps were the same as those in Example 6. In addition, the hPL solution is a solution containing fibrinogen although the hPL solution has been subjected to a fibrinogen reduction processing.

### Example 10

The method for producing a scaffold and method for culturing cells according to Example 10 were similar to those in Example 9, except for the following description. In Example 10, during the preparation of a cell suspension, a human platelet lysate was mixed with a mixture of DMEM and FBS so that a volume ratio of the human platelet lysate to the mixture of DMEM and FBS becomes 7 : 3 at a temperature of 37° C. The other steps were the same as those in Example 9.

### Example 11

The method for producing a scaffold and method for culturing cells according to Example 11 were similar to those in Example 9, except for the following description. In Example 11, during the preparation of a cell suspension, a human platelet lysate was mixed with a mixture of DMEM and FBS so that a volume ratio of the human platelet lysate to the mixture of DMEM and FBS becomes 4 : 6 at a temperature of 37° C. The other steps were the same as those in Example 9.

### Example 12

The method for producing a scaffold and method for culturing cells according to Example 12 were similar to those in Example 9, except for the following description. In Example 12, a human platelet lysate was used without using DMEM and FBS during the preparation of a cell suspension. The other steps were the same as those in Example 9.

### Reference Example 6

The method for producing a scaffold and method for culturing cells according to Reference Example 6 were similar to those in Example 9, except for the following description. In Reference Example 6, a human platelet lysate was not mixed with a mixture of DMEM and FBS during the preparation of a cell suspension. That is, the cell suspension was formed by introducing human mesenchymal stem cells from bone marrow into a mixture of DMEM and FBS. The other steps were the same as those in Example 9.

The results of culturing cells for Examples 9 to 12 and Reference Example 6 are shown in Table 4 below.

TABLE 4

Cell culture inside of the hydrogel

| | Introduction of hPL to the cell suspension into the hydrogel | Introdution of hPL into the medium | Number of seeded cells (cells) | Proliferat ion rate (times) | Culture days (days) | Doubling time (time) |
|---|---|---|---|---|---|---|
| Example 9 | 10% | Yes | 2.0×10⁴ | 2.5 | 9 | 163.4 |
| Example 10 | 30% | Yes | 2.0×10⁴ | 8.75 | 9 | 69.0 |
| Example 11 | 60% | Yes | 2.0×10⁴ | 15.2 | 9 | 55.0 |
| Example 12 | 100% | Yes | 2.0×10⁴ | 16.3 | 9 | 53.6 |
| Reference Example 6 | No | Yes | 2.0×10⁴ | 0.7 | 9 | - |

The proliferation rates of the cells in Examples 9 to 12 were each higher than that in Reference Example 6. Therefore, it has been found that the proliferation rate of cells is higher when a human platelet lysate was mixed with a cell suspension. In other words, it has been found that the scaffold obtained by adhering the plasma-derived or platelet-derived component or the fibrin-containing material to the inner surface of the hydrogel with the tube shape is more suitable for culturing cells.

Further, referring to Examples 9 to 12, it can be understood that the higher the concentration of the human platelet lysate in the cell suspension is when a scaffold is produced, the higher the proliferation rate of cells is and the shorter the doubling time is.

Further, when comparing between Examples 6 to 8 and Examples 10 to 12, it can be understood that by increasing the concentration of fibrinogen in the liquid medium to be used during culturing cells, the proliferation rates of cells becomes higher, and the doubling time becomes shorter.

When the scaffold having the hydrogel with the tube shape was produced and when mesenchymal stem cells were cultured by using the scaffold, it was not able to be confirmed that mesenchymal stem cells adhered onto the inner surface of the scaffold with the tube shape and proliferated, in a case of being treated with nattokinase that specifically degrades fibrin. That is, in the above-described Examples, it can be understood that at least fibrin contributes to the adhesion and proliferation of cells.

### Example 13

### Production of Scaffold

Example 13 will be described in detail. The scaffold in Example 13 is a scaffold in a form of a tube as shown in FIGS. 4 and 5. First, as the reagents, the following ones were prepared.
Sodium alginate (“I-1G” manufactured by KIMICA Corporation)
Human mesenchymal stem cells from bone marrow: hMSC-BM (“C-12974” manufactured by Promo Cell)
Medium for MSC (Mesenchymal Stem Cell Growth Medium 2 (Ready-to-use): C-28009)
Fibrinogen: human plasma-derived (JAN 4987481365186 manufactured by FUJIFILM Wako Pure Chemical Corporation)
Phosphate-buffered saline: PBS (JAN 4987481628489 manufactured by FUJIFILM Wako Pure Chemical Corporation)

First, a cell suspension, a sodium alginate solution, and a calcium chloride aqueous solution were prepared. The sodium alginate solution was generated by adding the above sodium alginate to a saline solution, and stirring them. The concentration of the sodium alginate to the saline solution was 0.99% by mass.

The cell suspension was adjusted as follows. First, phosphate-buffered saline (PBS) in which the above fibrinogen had been dissolved was prepared. The concentration of fibrinogen to the phosphate-buffered saline was 5 mg/mL at a temperature of 37° C. Next, a cell suspension was produced by introducing human mesenchymal stem cells from bone marrow into the fibrinogen-containing phosphate-buffered saline at a density of 10⁵ to 10⁶ cells/mL.

Next, a scaffold of a hydrogel with a tube shape covering the cell suspension was prepared in accordance with the method for preparing a scaffold shown in FIG. 7 and the description accompanying FIG. 7. That is, a flow of the above-described cell suspension, a flow of a sodium alginate solution around the flow of the cell suspension, and a flow of a calcium chloride aqueous solution around the flow of the sodium alginate solution were formed, and these flows were ejected into a saline solution (see also FIG. 7). The sodium alginate solution was crosslinked by being brought into contact with a calcium chloride aqueous solution, and an alginate gel was formed. In this way, a hydrogel having an elongated tube encasing the cell suspension was generated in a saline solution.

Next, the generated hydrogel with the elongated tube was transferred to a saline solution with the addition of thrombin, and left to stand at 37° C. for 30 minutes. The concentration of the thrombin to the saline solution may be around the degree of sufficiently converting the fibrinogen inside the hydrogel into fibrin.

According to the above steps, the scaffold having fibrin inside the hydrogel in the tube shape was formed. It is considered that the concentration of fibrin inside the hydrogel in a form of a tube roughly corresponds to the concentration of fibrinogen introduced into the inside of the hydrogel in a form of a tube.

Next, a scaffold having fibrin inside the hydrogel in a form of a tube was immersed in the above medium for MSC, and the human mesenchymal stem cells from bone marrow encapsulated inside the scaffold were cultured for 7 days. In addition, during culture, the medium for MSC was replaced every 2 to 3 days.

### Recovery of Cells

A method for recovering the cells cultured by using the above-described scaffold will be described. First, by an EDTA/PBS solution with the addition of nattokinase, fibrin is dissolved together with the hydrogel in a form of a tube. Subsequently, it was confirmed that the shape of the scaffold had collapsed sufficiently, and then the cells were recovered by centrifugation. After that, the number of recovered cells was measured with a hemocytometer.

### Example 14

The method for producing a scaffold and method for culturing cells according to Example 14 were similar to those in Example 13, except for the following description. In Examples 14, during the adjustment of a cell suspension, the concentration of fibrinogen to the phosphate-buffered saline was set so as to be 10 mg/mL at a temperature of 37° C. The other points were the same as those in Example 13. In Example 14, it is considered that the concentration of fibrin inside the hydrogel in a form of a tube roughly corresponds to the concentration of fibrinogen introduced into the inside of the hydrogel in a form of a tube.

### Example 15

The method for producing a scaffold and method for culturing cells according to Example 15 were similar to those in Example 13, except for the following description. In Examples 15, during the adjustment of a cell suspension, the concentration of fibrinogen to the phosphate-buffered saline was set so as to be 25 mg/mL at a temperature of 37° C. The other points were the same as those in Example 13. In Example 15, it is considered that the concentration of fibrin inside the hydrogel in a form of a tube roughly corresponds to the concentration of fibrinogen introduced into the inside of the hydrogel in a form of a tube.

### Example 16

The method for producing a scaffold and method for culturing cells according to Example 16 were similar to those in Example 13, except for the following description. In Examples 16, during the adjustment of a cell suspension, the concentration of fibrinogen to the phosphate-buffered saline was set so as to be 50 mg/mL at a temperature of 37° C. The other points were the same as those in Example 13. In Example 16, it is considered that the concentration of fibrin inside the hydrogel in a form of a tube roughly corresponds to the concentration of fibrinogen introduced into the inside of the hydrogel in a form of a tube.

### Reference Example 7

The method for producing a scaffold and method for culturing cells according to Reference Example 7 were similar to those in Example 13, except for the following description. In Reference Example 7, during the adjustment of a cell suspension, fibrinogen was not added to the phosphate-buffered saline. The other points were the same as those in Example 13. As a result, in Reference Example 7, fibrin did not precipitate inside the hydrogel in a form of a tube.

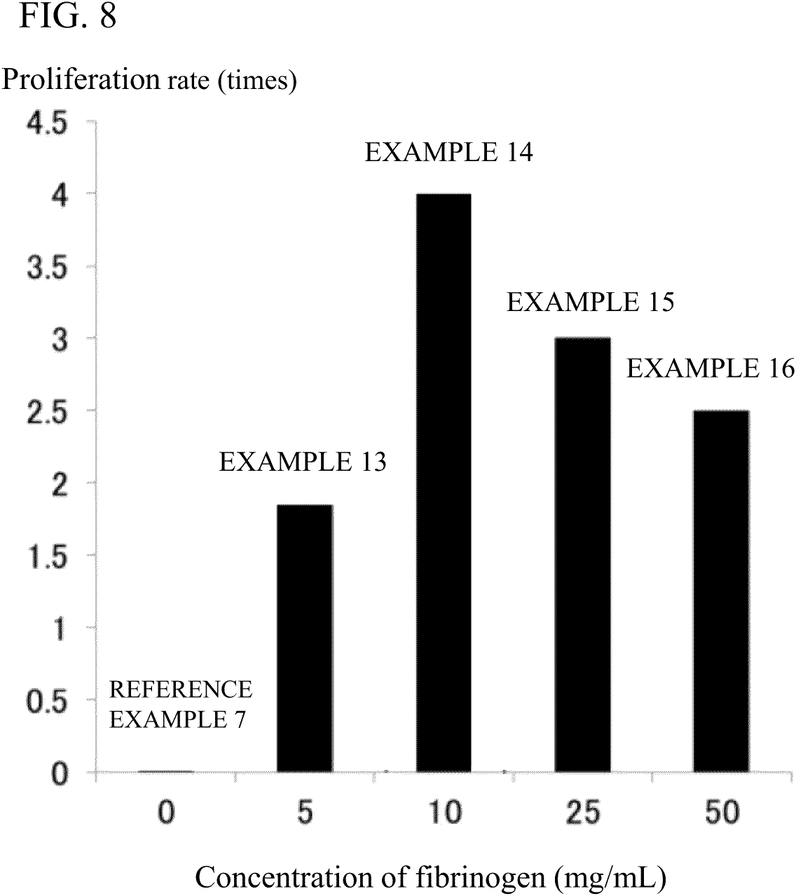

FIG. 8 is a graph showing proliferation rates of the cells cultured in Examples 13 to 16 and Reference Example 7. Further, in the graph of FIG. 8, the case where the fibrin concentration is 0 corresponds to Reference Example 7. In addition, the proliferation rate shown in FIG. 8 is a value calculated by dividing the number of cells recovered after 7 days by the number of cells at the time point when the scaffold was produced.

In Reference Example 7, cells inside the hydrogel in a form of a tube aggregated into round masses, and the number of cells decreased. On the other hand, in Examples 13 to 16, the proliferated cells were increased as compared with those in Reference Example 7. Consequently, it has been found that a scaffold obtained by allowing fibrin to adhere to the inside of the hydrogel in a form of a tube is more suitable for culturing cells. Further, it has been found that in a case where the concentration of fibrin inside the hydrogel in a form of a tube, in other words, the concentration of fibrinogen during the adjustment of a cell suspension is at least 5 to 50 mg/mL, the hydrogel is significantly superior to the hydrogel in a form of a tube without containing fibrinogen.

In addition, as described in Examples 9 to 12, in a case where fibrin is allowed to precipitate inside the hydrogel in a form of a tube by treating fibrinogen-containing phosphate-buffered saline with thrombin, it is sometimes difficult to uniformly coat the fibrin depending on the conditions. On the other hand, as described in Examples 6 to 12, in a case where a plasma-derived or platelet-derived component or a fibrin-containing material is allowed to adhere to the inside of the hydrogel in a form of a tube by using a human platelet lysate (hPL), the coating is easy, and the hydrogel is more suitable for culturing cells.

Accordingly, it is more preferable that by using a human platelet lysate (hPL) containing a plasma-derived or platelet-derived component, fibrinogen, fibrin, or a mixture thereof, the plasma-derived or platelet-derived component or a fibrin-containing material is allowed to adhere to the inside of the hydrogel in a form of a tube. However, as described in Examples 9 to 12, it should be noted that an embodiment in which fibrin is precipitated inside the hydrogel in a form of a tube by treating a solution containing fibrinogen with thrombin is also included within the scope of the present invention.

### Example 17

The method for producing a scaffold and method for culturing cells according to Example 17 were similar to those in Example 9, except for the following description. In Example 17, when the hydrogel is formed in the tube shape, the concentration of the sodium alginate to the saline solution was 0.99% by mass. In Example 17, the DMEM and the FBS were mixed so as to have a volume ratio of 9 : 1 at a temperature of 37° C. Further, the above human platelet lysate was mixed with the mixture of DMEM and FBS to form a cell suspension so that a volume ratio of the human platelet lysate to the mixture of the DMEM and the FBS becomes 100 : 6 at a temperature of 37° C. Therefore, the concentration of the hPL in the suspension is 6% by volume at a temperature of 37° C. The density of the cells seeded in the cell suspension in Example 17 was 1.0×10⁵ cells/mL when the hydrogel is formed in the tube shape. The other steps were the same as those in Example 9.

The scaffold of a hydrogel with the tube shape covering the cell suspension was prepared in accordance with the method for preparing a scaffold shown in FIG. 7. The human mesenchymal stem cells were cultured in the hydrogel with the tube shape for nine days as described in Example 9.

### Example 18

The method for producing a scaffold and method for culturing cells according to Example 18 were similar to those in Example 17, except for the following description. In Example 18, the above human platelet lysate was mixed with the mixture of DMEM and FBS to form a cell suspension so that a volume ratio of the human platelet lysate to the mixture of the DMEM and the FBS becomes 100 : 8 at a temperature of 37° C. Therefore, the concentration of the hPL in the suspension is 8% by volume at a temperature of 37° C. The other steps were the same as those in Example 17.

### Example 19

The method for producing a scaffold and method for culturing cells according to Example 19 were similar to those in Example 17, except for the following description. In Example 19, the above human platelet lysate was mixed with the mixture of DMEM and FBS to form a cell suspension so that a volume ratio of the human platelet lysate to the mixture of the DMEM and the FBS becomes 100 : 10 at a temperature of 37° C. Therefore, the concentration of the hPL in the suspension is 10% by volume at a temperature of 37° C. The other steps were the same as those in Example 17.

### Reference Example 8

The method for producing a scaffold and method for culturing cells according to Reference Example 8 were similar to those in Example 17, except for the following description. In Reference Example 8, a human platelet lysate was not mixed with a mixture of DMEM and FBS during the preparation of a cell suspension. That is, the cell suspension was formed by introducing human mesenchymal stem cells from bone marrow into a mixture of DMEM and FBS. The other steps were the same as those in Example 17.

The results of culturing cells for Examples 17 to 19 are shown in Table 5 below. The proliferation rates of the cells in Examples 17 to 19 were each higher than that in Reference Example 8. Therefore, it has been found that the proliferation rate of cells is higher when a human platelet lysate was mixed with a cell suspension. In other words, it has been found that the scaffold obtained by adhering the plasma-derived or platelet-derived component or the fibrin-containing material to the inner surface of the hydrogel with the tube shape is more suitable for culturing cells.

TABLE 5

Cell culture inside of the hydrogel

| | Introduction of hPL to the cell suspension into the hydrogel | Introdution of hPL into the medium | Number of seeded cells (cells) | Proliferat ion rate (times) | Culture days (days) | Doubling time (time) |
|---|---|---|---|---|---|---|
| Example 17 | 6% | Yes | 1.0×10⁵ | 1.5 | 9 | 369.3 |
| Example 18 | 8% | Yes | 1.0×10⁵ | 4.5 | 9 | 99.5 |
| Example 19 | 10% | Yes | 1.0×10⁵ | 12.8 | 9 | 58.7 |
| Reference Example 8 | No | Yes | 1.0×10⁵ | 0.2 | 9 | - |

Further, referring to Examples 17 to 19, it can be understood that the higher the concentration of a human platelet lysate in a cell suspension is when the scaffold is produced, the higher the proliferation rate of cells is and the shorter the doubling time is.

From the results in Examples 9 to 12 and Examples 17 to 19, the proportion of the hPL in a cell suspension at a temperature of 37° C. is, for example, 6% by volume or more, preferably 8% by volume or more, and more preferably 10% by volume or more.

Further, an animal or mammalian platelet lysate (animal PL or mammalian PL) may also be used in place of the human platelet lysate (hPL). That is, the plasma-derived or platelet-derived component, the fibrin-containing material or fibrin polymer can be coated on the inner surface or the outer surface of the hydrogel by using an animal or mammalian platelet lysate (animal PL or mammalian PL) containing the above-described plasma-derived or platelet-derived component, fibrinogen, fibrin or a mixture thereof.

According to the above embodiments and/or examples, the coating including plasma-derived or platelet-derived component, a fibrin-containing material or fibrin polymer can be provided the inside or the outside of the hydrogel. Furthermore, the adherent cells can be adhered to an entire of the coating provided on the inner surface and/or outer surface of the hydrogel by culturing the adherent cells.

Preferably, the coating including plasma-derived or platelet-derived component, a fibrin-containing material or fibrin polymer has a thickness of less than 10 µm. More preferably, the coating has the thickness of less than 9 µm or less than 8 µm. The coating with such small thickness can be formed by the methods previously described.

It should be noted that the term “scaffold” as used herein is a concept that does not contain cell(s) as a constitution.

It can be understood that at least the following inventions are specified in the present specification, from the above-described embodiments and/or Examples, the description added below, and the like.

### Appendix 1

A scaffold for culturing cells, including:
a hydrogel; and
a plasma-derived or platelet-derived component or a fibrin-containing material, adhered to the hydrogel.

The plasma-derived or platelet-derived component is as described above. The fibrin-containing material may be fibrin itself, for example, a fibrin polymer, or may be a mixture of fibrin and other polymers.

### Appendix 2

The scaffold according to Appendix 1, wherein

the hydrogel has a string shape, a tube shape, a sphere shape, or a spherical shell shape.

### Appendix 3

The scaffold according to Appendix 2, wherein

the plasma-derived or platelet-derived component or the fibrin-containing material is provided an inside or outside of the hydrogel.

### Appendix 4

The scaffold according to any one of Appendixes 1-3, wherein

the plasma-derived or platelet-derived component or the fibrin-containing material contains a human platelet lysate-derived component.

### Appendix 5

The scaffold according to any one of Appendixes 1-4, wherein the hydrogel contains an alginate gel.

### Appendix 6

The scaffold according to any one of Appendixes 1-5, wherein

the hydrogel contains an alginate gel, and a gelatin mixed with the alginate gel.

### Appendix 7

A scaffold for culturing cells, including:
a hydrogel; and
a fibrin polymer provided on the hydrogel.

The hydrogel may have, for example, string shape, a tube shape, a sphere shape, or a spherical shell shape. In this case, the fibrin polymer may be arranged on the outer surface of a hydrogel in a form of a string or a sphere, or on the inside of a hydrogel in a form of a tube or a spherical shell.

### Appendix 8

The scaffold described in Appendix 7, including a fibrin polymer and albumin provided on the hydrogel.

The hydrogel may have, for example, a string shape, a tube shape, a sphere shape, or a spherical shell shape. In this case, the fibrin polymer and albumin may be provided on the outer surface of a hydrogel in a form of a string or a sphere, or on the inside of a hydrogel in a form of a tube or a spherical shell.

### Appendix 9

A cell culture construct, including:
the scaffold according to any one of Appendixes 1 to 8; and
cells adhered to the scaffold.

Here, the type of the cells adhered to the scaffold is as already listed.

### Appendix 10

The cell culture construct according to Appendix 9, wherein the cell is an adherent cell.

### Appendix 11

The cell culture construct according to Appendix 10, wherein the adherent cell is a mesenchymal stem cell.

### Appendix 12

A method for culturing a cell, comprising,

culturing a cell by adhering the cell to the scaffold according to any one of Appendixes 1 to 8.

### Appendix 13

A method for producing a scaffold for culturing cells, including,

bringing a suspension containing a plasma-derived or platelet-derived component, fibrinogen or fibrin, or a mixture thereof into contact with a hydrogel.

Preferably, the suspension contains fibrinogen. In this case, the amount of fibrinogen to be introduced in a suspension, for example, the amount of fibrinogen to be introduced to phosphate-buffered saline may be, for example, 1 mg/mL or more, preferably 3 mg/mL or more, and more preferably 5 mg/mL or more. The upper limit of the amount of fibrinogen to be introduced is not particularly limited, but may be, for example, 300 mg/mL. In addition, a fibrin polymer may be formed by acting thrombin on fibrinogen in a suspension.

### Appendix 14

The method for producing a scaffold according to Appendix 13, wherein the suspension contains fibrinogen and albumin.

### Appendix 15

The method for producing a scaffold according to Appendix 13 or 14, wherein the method comprises:
turning a hydrogel precursor into a gel to form the alginate gel; and
immersing the alginate gel in the suspension.

### Appendix 16

The method for producing a scaffold according to Appendix 13 or 14, wherein the method comprises:
flowing a hydrogel precursor around a suspension while flowing the suspension; and
turning the hydrogel precursor into a gel to form the hydrogel which has a tube shape covering the suspension.

### Appendix 17

The method for producing a scaffold according to any one of Appendixes 13 to 16, wherein the suspension contains a human platelet lysate.

### Appendix 18

The method for producing a scaffold according to any one of Appendixes 13 to 17, wherein the hydrogel precursor contains an alginate solution.

### Appendix 19

A cell population obtained by removing the scaffold from the cell culture construct according to any one of Appendixes 9 to 11.

### Appendix 20

A biological substance generated by at least one of the cell adhered to the scaffold in the cell culture construct according to any one of Appendixes 9 to 11, an cell recovered from the scaffold in the cell culture construct according to any one of Appendixes 9 to 11, and the cell population according to Appendix 19.

The biological substance may be any substance generated by cells. The biological substance may be, for example, a macromolecule such as a nucleic acid, a protein, or a polysaccharide. Such a biological substance can be generated during culturing the cells or cell population in the above-described cell culture.

Further, the cells in a state of adhering to a scaffold includes not only the cells adhered onto the outer surface of a scaffold, but also the cells in a state of being encapsulated in a hydrogel that forms the scaffold. In a case where the hydrogel is, for example, in a form of a tube or a spherical shell, the generated substance includes a substance generated by the cells encapsulated in a hydrogel in a form of a tube or a spherical shell.

As described above, the contents of the present invention have been disclosed through the embodiments and/or examples. However, it should not be understood that the description and drawings forming a part of the present disclosure limit the present invention. From this disclosure, various alternative embodiments, examples, and operation techniques will be apparent to those skilled in the art. Therefore, the technical scope of the present invention is determined only by the matters specifying the invention according to the claims that are appropriate from the above description.

1. A scaffold for culturing an adherent cell, comprising:
a hydrogel having a string shape, a tube shape, a sphere shape or a spherical shell shape; and
a coating including plasma-derived or platelet-derived component, a fibrin-containing material or fibrin polymer which is provided an inside or an outside of the hydrogel, wherein
the coating has a thickness of less than 10 µm.

2. The scaffold according to claim 1, wherein
the hydrogel has the tube shape or the spherical shell shape,
the plasma-derived or platelet-derived component, the fibrin-containing material or the fibrin polymer is provided the inside of the hydrogel.

3. The scaffold according to claim 1, wherein
the plasma-derived or platelet-derived component, the fibrin-containing material or the fibrin polymer contains a platelet lysate-derived component.

4. The scaffold according to claim 1, wherein
the coating has the fibrin polymer and albumin.

5. A cell culture construct, comprising:
the scaffold according to claim 1; and
an adherent cell adhered to the scaffold.

6. The cell culture construct according to claim 5, comprising adherent cells adhered to an entire surface of the coating.

7. The cell culture construct according to claim 5, wherein
the adherent cell is a mesenchymal stem cell.

8. A cell population obtained by removing the scaffold from the cell culture construct according to claim 5.

9. A biological substance generated by at least one of the cell adhered to the scaffold in the cell culture construct according to claim 5, an cell recovered from the scaffold in the cell culture construct according to claim 5, and the cell population obtained by removing the scaffold from the cell culture construct according to claim 5.

10. A method, comprising,
culturing an adherent cell while adhering the adherent cell to the scaffold according to claim 1.

11. The method according to claim 10, comprising
culturing the adherent cells to adhere to an entire surface of the coating.

12. A method for producing a scaffold for culturing an adherent cell, comprising,
forming a coating including plasma-derived or platelet-derived component, a fibrin-containing material or fibrin polymer an inside or an outside of a hydrogel having a string shape, a tube shape, a sphere shape or a spherical shell shape by contacting a suspension containing a plasma-derived or platelet-derived component, fibrinogen, fibrin or a mixture thereof with the hydrogel gel.

13. The method for producing a scaffold according to claim 12, wherein the method comprises:
turning a hydrogel precursor into the hydrogel gel having the string shape, the tube shape, the sphere shape or the spherical shell shape; and
forming the coating the outside of the hydrogel by immersing the hydrogel gel in the suspension.

14. The method for producing a scaffold according to claim 12, wherein the method comprises:
flowing a hydrogel precursor around the suspension while flowing the suspension including an adherent cell; and
turning the hydrogel precursor into the hydrogel having a tube shape surrounding the suspension, and forming the coating the inside of the hydrogel by precipitation of components in the suspension.

15. The method for producing a scaffold according to claim 12, wherein
the suspension contains a platelet lysate.

16. The method for producing a scaffold according to claim 14, wherein
the suspension contains fibrinogen, and
a concentration of the fibrinogen in the suspension is 5 mg/mL or more at a temperature of 37° C.

17. The method for producing a scaffold according to claim 14, wherein
the suspension contains a hPL.

18. The method for producing a scaffold according to claim 17, wherein
a concentration of the hPL in the suspension is 6% by volume or more at a temperature of 37° C.

19. The method for producing a scaffold according to claim 17, wherein
a concentration of the hPL in the suspension is 10% by volume or more at a temperature of 37° C.

20. The method for producing a scaffold according to claim 12, wherein
the adherent cell is a mesenchymal stem cell.
